(19) United States
(12) Patent Application Publication
(10) Pub. No.: US 2021/0251901 A1
(43) Pub. Date: Aug. 19, 2021

(54) Fine Dry Particulate Adenosine Compositions and Topical Formulations Including the Same

(71) Applicant: Laboratory Skin Care, Inc., Redwood City, CA (US)

(72) Inventor: Zahra Mansouri, Redwood City, CA (US)

(21) Appl. No.: 17/181,483

(22) Filed: Feb. 22, 2021

Related U.S. Application Data
(63) Continuation of application No. 16/600,015, filed on Oct. 11, 2019, now abandoned.
Application No. 16/600,015 is a continuation of application No. 15/530,588, filed on Feb. 2, 2017, now abandoned.
Application No. 15/530,588 is a continuation of application No. 14/239,095, filed on May 22, 2014, now abandoned.
Application No. 16/600,015 is a continuation-in-part of application No. 16/429,816, filed on Jun. 3, 2019, now abandoned.
Application No. 16/429,816 is a continuation of application No. 15/619,253, filed on Jun. 9, 2017, now Pat. No. 10,350,168.
Application No. 15/619,253 is a continuation of application No. 14/259,010, filed on Apr. 22, 2014, now Pat. No. 9,707,177.
Application No. 14/259,010 is a continuation of application No. 13/858,677, filed on Apr. 8, 2013, now abandoned.
Application No. 13/858,677 is a continuation of application No. 12/742,235, filed on Apr. 12, 2011, now Pat. No. 8,445,002.
(60) Provisional application No. 61/524,295, filed on Aug. 16, 2011.
(60) Provisional application No. 61/176,057, filed on May 6, 2009.

(51) Int. Cl. A61K 9/16; A61K 9/00; A61K 31/70; A61K 9/14; A61K 9/06; A61K 47/02; A61K 31/7076

(52) U.S. Cl. CPC A61K 9/1611; A61K 9/0014; A61K 31/70; A61K 31/7076; A61K 9/06; A61K 47/02; A61K 9/143

(57) ABSTRACT

Fine dry particulate adenosine compositions suitable for use in topical formulations, as well as methods of making the same, are provided. In the dry particulate adenosine composition, the adenosine active agent is associated with the particles, e.g., via entrapment in the pores of the particles and/or ionic binding and/or non-covalent binding to the surface of the particles and/or loosely associated with the particles. Also provided are topical formulations which include the dry particulate adenosine compositions of the invention, and methods of using the same.

### CROSS-REFERENCE TO RELATED APPLICATIONS

Pursuant to 35 U.S.C. § 119 (e), this application claims priority to the filing date of U.S. Provisional Patent Application Ser. No. 61/524,295 filed Aug. 16, 2011; the disclosure of which application is herein incorporated by reference.

### INTRODUCTION

Skin includes a surface layer, known as the epidermis, and a deeper connective tissue layer, known as the dermis. The epidermis undergoes continuous turnover as the outermost cells are exfoliated and replaced by cells that arise from inner dermal layers. The dermis is composed of a variety of cell types, including fibroblasts.

Skin thickness begins to decline in humans after the age of 20 as the dermis becomes thinner and the number of skin fibroblasts declines. As skin ages, or is exposed to UV light and other environmental insults, changes in the underlying dermis can lead to the functional and morphological changes associated with damaged skin. Decreases in the abundance and function of products of the fibroblasts, which include collagen and proteoglycans, are believed to play major roles in wrinkled and damaged skin.

Adenosine delivery to the skin is beneficial for the prevention and rejuvination of skin disorders, and is thought to exert its beneficial activity by stimulating active cells.

### SUMMARY

Fine dry particulate adenosine compositions suitable for use in topical formulations, as well as methods of making the same, are provided. Aspects of the methods include combining an amount of nanoporous calcium particles and one or more adenosine active agents in a manner sufficient to produce a dry particulate adenosine active agent composition. In the dry particulate adenosine composition, the adenosine active agent is associated with the particles, e.g., via entrapment in the pores of the particles and/or ionic binding and/or non-covalent binding to the surface of the particles and/or loosely associated with the particles. Also provided are examples of topical formulations which include the dry particulate adenosine compositions of the invention, and methods of using the same.

### BRIEF DESCRIPTION OF THE FIGURE

FIG. 1 provides a graphical representation of a dermal delivery assay described in the experimental section below.

### DETAILED DESCRIPTION

Fine dry particulate adenosine compositions suitable for use in topical formulations, as well as methods of making the same, are provided. Aspects of the methods include combining an amount of nanoporous calcium particles and one or more adenosine active agents in a manner sufficient to produce a dry particulate adenosine active agent composition. In the dry particulate adenosine composition, the adenosine active agent is associated with the particles, e.g., via entrapment in the pores of the particles and/or ionic binding and/or non-covalent binding to the surface of the particles and/or loosely associated with the particles. Also provided are examples of topical formulations which include the dry particulate adenosine compositions of the invention, and methods of using the same.

Before the present invention is further described, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Methods recited herein may be carried out in any order of the recited events which is logically possible, as well as the recited order of events.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Methods of Making Fine Dry Particulate Actives

As summarized above, aspects of the invention include methods of making fine dry particulate adenosine compositions, where the methods include combining an amount of nanoporous calcium particles (e.g., calcium phosphate particles) and one or more adenosine active agents in a manner sufficient to produce a dry particulate adenosine composition. As reviewed above, in the dry particulate adenosine composition, the active agent is associated with the particles, e.g., via entrapment in the pores of the particles and/or ionic binding and/or non-covalent binding to the surface of the particles and/or loosely associated with the particles. In practicing methods according to embodiments of the invention, nanoporous calcium particles and one or more adenosine active agents are combined in the presence of a suitable aqueous solvent system under conditions sufficient for the active agent(s) to enter internal space of the particles and/or ionically bind and/or covalently bind and/or associate with the surface of the particles. Before further describing the method steps, the particles, active agents and solvent systems employed in certain embodiments of the methods are reviewed in greater detail.

### Nanoporous Calcium Particles

Particles employed in methods of the invention are nanoporous phosphate particles. By “nanoporous” is meant that the particles have a porosity of 30% or more, such as 40% or more, including 50% or more, where the porosity may range from 30% to 85%, such as from 40% to 70%, including from 45% to 55%, as determined using a mercury intrusion porosimeter porosity determination protocol as described in ASTM D 4284-88 “Standard Test Method for Determining Pore Volume Distribution of Catalysts by Mercury Intrusion Porosimetry”. Porosity is also described by “pore volume (ml/g)” and in such instances many range from 0.1 ml/g to 2.0 ml/g. In some cases, the particles have a porosity such that their internal surface area ranges from 10 m²/g to 150 m²/g, such as from 20 m²/g to 100 m²/g, including 30 m²/g to 80 m²/g, as determined using a BET gas adsorption surface area determination protocol as described in ASTM D3663-03 Standard Test Method for Surface Area of Catalysts and Catalyst Carriers. The pore diameter may vary, ranging in certain instances from 2 to 100 nm, such as 5 to 80 nm, including 10 to 60 nm. In addition, the particles may have a tapping density ranging from 0.2 g/cm³ to 0.5 g/cm³, such as from 0.25 g/cm³ to 0.45 g/cm³, including from 0.3 g/cm³ to 0.4 g/cm³. The tap density can be measured by using standard ASTM WK13023—New Determination of Tap Density of Metallic Powders by a Constant Volume Measuring Method.

In some instances, the particles are rigid particles which are uniform and spherical in shape. By “rigid” is meant that the particles are hard, such that they are not pliant. By “uniform” is meant that the shape of the particles does not vary substantially, such that the particles have substantially the same spherical shape. The term “spherical” is employed in its conventional sense to mean a round body whose surface is at all points substantially equidistant from the center. Of interest in certain embodiments are calcium particles in which the median diameter is 20 μm or less, such as 10 μm or less, including 5 μm or less, where in some instances the medium diameter is 4 μm or less, such as 3 μm or less, including 2 μm or less.

The particles are, in some instances, chemically pure. By chemically pure is meant that the particles are made up of substantially one type of compound, e.g., a calcium compound, such as a calcium phosphate mineral. Of interest as porous particles are calcium containing particles, such as calcium containing particles that are made of a molecule that includes calcium cation and a suitable anion, e.g., carbonate, phosphate, etc. In some instances, the particles are calcium carbonate particles, such as but not limited to the calcium carbonate particles disclosed in U.S. Pat. Nos. 5,292,495 and 7,754,176. In some instances, the calcium phosphate particles are made up of a calcium phosphate that is described by the molecular formula Ca₁₀(PO₄)₆(OH)₂.

In some instances, the particles are ceramic particles. By ceramic is meant that the particles are produced using a method which includes a step of subjecting the particles to high temperature conditions, where such conditions are illustrated below. High temperatures may range from 200 to 1000° C., such as 300 to 900° C. and including 300 to 800° C. In some embodiments, the particles have a compression rupture strength ranging from 20 to 200 MPa, such as from 50 to 150 MPa, and including 75 to 90 MPa, as determined using a SHIMADZU MCT-W500 micro-compression testing machine particle strength determination protocol with a particle sintered at temperature of 400° C. to 900° C., as described in European Patent EP1840661. In some embodiments, the particles are biodegradable, by which is meant that the particles degrade in some manner, e.g., dissolve, over time under physiological conditions. As the particles of these embodiments are biodegradeable under physiological conditions, they at least begin to dissolve at a detectable rate under conditions of pH of 5.8 or less, such as 5.5 or less, e.g., 5.3 or less, including 5 or less, e.g., 4.9 or less.

The uniform, rigid, spherical, nanoporous calcium phosphate particles employed in embodiments of the methods may be prepared using any convenient protocol. In one protocol of interest, the particles are manufactured by spray drying a slurry which includes nanoporous calcium phosphate (e.g., hydroxyapatite) crystals (which may range from 2 nm to 100 nm size range) to produce uniform spherical nanoporous calcium phosphate particles. The resultant particles are then sintered for a period of time sufficient to provide mechanically and chemically stable rigid spheres. In this step, the sintering temperatures may range from 100° C. to 1000° C., such as 200° C. to 1000° C., such as 300° C. to 900° C. and including 300° C. to 800° C. for a period of time ranging from 1 hour to 10 hours, such as 2 hours to 8 hours and including 3 hours to 6 hours.

In some instances, the nanoporous calcium particles may be pre-treated. Pretreated particles may be prepared via a number of different protocols. In some instances, the particles may be neutralized with a pH adjuster, e.g., such as an acid. The pH may be adjusted to optimum range, which may be specific to the active agent, when necessary. Examples of pH adjusters of interest include weak or strong acids such as hydrochloric acid, glycolic acid, phosphoric acid, lactic acid and citric acid and others. In some instances, the particles may be pretreated with a phosphate salt, such as sodium phosphate or pretreated with a calcium salt, such as calcium chloride. In some instances, a mixture of buffering system is used such as sodium citrate and citric acid or calcium chloride and lactic acid. Where desired, any salts produced during this protocol may be removed, e.g., via filtering or decanting. Further details regarding pretreatment protocols of interest for nanoporous calcium phosphate particles may be found in U.S. Provisional Application Ser. No. 61/327,633.

### Adenosine Active Agent

The term “adenosine active agent” refers an agent that has adenosine activity. Examples of adenosine active agents include, but are not limited to: adenosine (beta-Adenosine; Adenoscan; Adenocor; Nucleocardyl; 9-beta-D-Ribofuranosyl-9H-purin-6-amine; 9-beta-D-Ribofuranosyladenine; Adenine Riboside; Adenocard; beta-d-Adenosine; Adenine-9beta-d-Ribofuranoside; Boniton; Myocol; Sandesin; 1-(6-amino-9H-purin-9-yl)-1-deoxy-beta-d-Ribofuranose; Adenine nucleoside; Adenosin; 9-beta-d-Ribofuranosyl-9H-Purin-6-amine; 9-beta-D-Arabinofuranosyladenine; 6-Amino-9beta-D-Ribofuranosyl-9H-purine; 9-beta-D-Ribofuranosidoadenine; 9-beta-D-Ribofuranosyl-9H-purin-6-amine); adenosine monophosphate (AMP), adenosine diphosphate (ADP) and adenosine triphosphate (ATP), as well as analogs thereof. Analogs of interest include, but are not limited to: 2′-deoxyadenosine; 2′, 3′-isopropoylidene adenosine; toyocamycin; 1-methyladenosine; N-6-methyladenosine; adenosine N-oxide; 6-methylmercaptopurine riboside; 6-chloropurine riboside, 5′adenosine monophosphate, 5′-adenosine diphosphate, or 5′-adenosine triphosphate. Additional adenosine active agents of interest include, but are not limited to: phenylisopropyl-adenosine (“PIA”), 1-Methylisoguanosine, ENBA (S(—), N⁶-Cyclohexyladenosine (CHA), N⁶-Cyclopentyladenosine (CPA), 2-Chloro-N⁶-cyclopentyladenosine, 2-chloroadenosine, and adenosine amine congener (ADAC), 2-p-(2-carboxy-ethyl) phenethyl-amino-5′-N-ethylcarboxamido-adenosine (CGS-21680), N-ethylcarboxamido-adenosine (NECA) and napthyl-substituted aralkoxyadenosine (SHA-082), 5′ (N-Cyclopropyl)-carboxamidoadenosine, DPMA (PD 129,944), Metrifudil, 2-Chloroadenosine, N⁶-Phenyladenosine, and N⁶-Phenylethyladenosine; 2-Phenylaminoadenosine and MECA.

### Solvent System

The solvent system may be made up of a single solvent or two or more different solvents, where the particular solvent or solvents making up the solvent system may be selected based on the nature of active agent to be complexed with the particles. In some instances, the solvent system is aqueous, and may be 100% water, or water in combination with one or more additional solvents, including polar and non-polar solvents, which may be organic or inorganic, as desired.

### Fabrication of Dry Particulate Actives

As summarized above, in preparing dry particulate actives in accordance with embodiments of the invention, the active agent(s), nanoporous calcium phosphate particles and solvent system are combined to produce a calcium phosphate particles/active agents mixture. The various components may be combined using any convenient protocol. In some instances, the active agent(s) is first dissolved in the solvent system, and then the resultant active agent solution is combined with an amount of calcium phosphate particles. In yet other instances, the calcium phosphate particles are combined first with the solvent system, and then the active agent is added to produce the calcium phosphate particles/active agents mixture.

The active agent(s) and solvent system may be combined using any protocol sufficient to produce the desired mixture solution. In some instances, the active agent(s) and solvent system are combined with agitation. Agitation may be provided using any convenient protocol, e.g., stir bar, agitation blade, propeller, etc. The temperature at which the active is combined with the solvent system and dissolved therein may vary, and may be below room temperature, at room temperature or above room temperature. The specific temperature at which the combination of active agent and solvent is carried out may be chosen based on the nature of the active agent (such that a temperature is chosen that will not inactivate the active agent) as well as the properties of the solvent system, e.g., melting point, boiling point, etc. In some instances, the temperature ranges from just above 0° C. to 200° C. In some instances, the temperature ranges from 4 to 25° C., e.g., 5 to 10° C. In some instances, the temperature is above room temperature, e.g., 35 to 60° C., e.g. 40 to 45° C., 50 to 55° C., or higher. In some instances, the temperature ranges from 65 to 150° C., e.g. 70 to 85° C., 90 to 105° C., 120 to 135° C. or higher. In some instances, the temperature ranges from 5 to 80° C., such as 5 to 75° C., e.g., 10 to 65° C., e.g., 20 to 60° C.

The amount of active agent that is dissolved in the solvent system may be selected based on the solubility of the active agent in the solvent system and/or based on the amount of calcium phosphate particles to be used. In some instances, the amount of active agent relative to the calcium phosphate particles is 0.1% by weight or more, such as 10% by weight or more, such as 20% by weight or more, such as 30% by weight or more, such as 40% by weight or more, such as 60% by weight or more, such as 70% by weight or more, such as 80% by or more, such as 90% by weight or more, including 100% by weight or more, including 1000% by weight or more. In some instances, the weight ratio of active agent(s) to calcium phosphate particles ranges from 0.01:10, 0.1:1, 1:1 and 1:0.1. In some instances, the weight ratio of active agent(s) to calcium particles ranges from 0.5:1.0 to 5:1, where in some instances the ratio is 1:1.

Following preparation of the active agent solution, e.g., as described above, a suitable amount of calcium phosphate particles (which may or may not be pre-treated, e.g., as described and referenced above) is combined with the solution. In some instances, the calcium phosphate particles that are combined with the active agent solution are dry. In some instances, the methods include wetting an initial amount of nanoporous calcium phosphate particles with a solvent system, where the solvent system may be the same as or different from that used to prepare the active agent solution, e.g., as described above.

The particles (either dry or wetted as described above) may be combined with a solution of an active agent present in a solvent system, e.g., as described above, to produce a liquid composition that includes particles and an active agent(s) in a solvent system, which composition may be referred to herein as an active agent mixture. The active agent solution and particles (dry or wetted, as desired) may be mixed using any convenient protocol, e.g., with agitation (such as described above), to produce a liquid composition that includes both the particles and the active agent in a solvent system. This mixing step lasts for a time sufficient to produce the desired mixture, and in some instances ranges in length from 1 minute to 600 minutes, such as 5 minutes to 300 minutes. In certain instances, the nanoporous calcium phosphate particles and active agent(s) solution are combined under negative pressure. When combined under negative pressure, pressures of interest may vary and in some instances range from 0.001 torr to 1 torr, such as 0.01 torr to 0.1 torr and including 0.05 torr to 0.5 torr.

Following preparation of the mixture, the solvent system is dried off from the active agent mixture to produce the desired fine dry particulate active. Drying may be accomplished using any convenient protocol, where protocols of interest include, but are not limited to: maintaining at elevated temperatures sufficient to evaporate the solvent. Drying methods of interest include, but are not limited to: drying by heat convection, such as spray drying, air flow drying, fluid bed drying, and super-heated steam drying, or drying by heat conduction, such as vacuum drying, freeze drying, rotary drum drying, and rotary vacuum drying or drying by heat radiation, such as infrared heat drying and microwave drying, or heat radiation with other electromagnetic waves, and or other methods such as super critical drying, etc. Combinations of various protocols may be employed, as desired. Following separation of the solvent, the resultant dry product may be further processed as desired, e.g., the product may be grinded, milled (e.g., via ball mill, hammer mill, jet impact mill, wet impact mill, etc.), sieved (e.g., with or without vibration, subjected to air-flow or jet-flow classification), etc., as desired, to produce a fine dry particulate active.

As indicated above, the active compositions of the invention may be characterized by having a single active agent associated with given calcium particles, or two or more active agents (e.g., three or more active agents, four or more, five or more) different active agents associated with the same calcium particles.

The above fabrication protocol results in the production of a fine dry particulate adenosine active of the invention. In the resultant dry powder active agent is present inside of the particles, and/or bound to the particles, covalently or ionically, and/or on the surface of the particles, and/or tightly associated with the particles and loosely associated with the particles. The amount of active agent component (which is made up of one or more distinct active agents) that is bound or associated with calcium phosphate particles may vary depending on the particular active agent(s). The resultant active particulate has a distribution of diameter of the particles, where in some instances the majority (such as 60% or more, 75% or more, 90% or more, 95% or more) of the particles have diameters that range from 0.01 to 100 μm, such as from 0.01 to 20 μm, such as from 0.1 to 10 μm, and including from 0.1 to 2 μm.

In some instances, the amount of active agent relative to the calcium particles ranges from 1% or less by weight to 500% by weight or more, e.g., in some instance being 50% by weight or more, such as 60% by weight or more, such as 70% by weight or more, such as 80% by or more, such as 90% by weight or more, including 100% by weight or more, such as 150% by weight or more, e.g., 500% by weight or more, including 1000% by weight or more. In some instances, the weight ratio of active agent(s) to calcium particles ranges from 0.5:1.0 to 5:1, e.g., 0.1 to 1 to 1:0.1, where in some instances the ratio is 1:1.

Depending on the nature of the resultant active to be employed, the protocols may or may not include a step of coating the resultant active powder. Coating materials (which may include one or more coating material) of interest are those that preserve the association of the active agent with the calcium phosphate particles in various formulations, e.g. formulations designed for topical application to the skin. Suitable coating agents include agents that are physiologically acceptable and are solid at room temperature and are suitable for application to the skin. The coating material component may be a single material or a combination of two or more materials, e.g., where the combination provides for one or more desirable properties. Materials that find use as coating materials include, but are not limited to waxes, butters, etc. Coatings materials of interest and methods for their use are further described in U.S. patent application Ser. No. 12/565,687; the disclosure of which is herein incorporated by reference.

### Topical Formulations

Aspects of the invention further include topical formulations that are configured for application to a topical site of a human subject. Topical formulations of the invention are for applications such as mucosal surface or keratinized skin surface of a mammalian subject, such as a human subject. By mucosal surface is meant a location of a subject that includes a mucosal membrane, such as the inside of the mouth, in the inside of the nose, etc. By keratinized skin surface is meant a skin location of a subject, i.e., a location of the external covering or integument of an animal body. Because the topical formulations of the invention are formulated for delivery to topical location, they are formulated so as to be physiologically compatible with the topical location for which they are formulated. Accordingly, when contacted with the target keratinized skin surface for which they are formulated, the topical compositions do not cause substantial, if any, physiological responses (such as inflammation or irritation) that would render the use of the topical compositions unsuitable for topical application. Topical formulations of the invention include: (a) an amount of the actives (which may or may not be stabilized); and (b) a topical delivery vehicle.

As indicated above, the topical compositions include an amount of the fine dry particulate active present in a topical delivery vehicle. The amount of fine dry particulate active that is present in the delivery composition and therefore combined with a delivery vehicle may vary. In some embodiments, the amount of fine dry particulate active present in the delivery vehicle ranges from 0.01 mg/g to 500 mg/g, such as 0.01 to 250 mg/g, such as 0.1 to 200 mg/g, e.g., 1 to 100 mg/g, including 10 to 50 mg/g fine dry particulate active per gram of delivery vehicle. In certain embodiments the fine dry particulate active are present in compositions in an amount ranging from about 0.001% or more by weight, such as 0.01%, or 0.05%, or 1% or more, 5% or more, 10% or more, 15% or more, 25% or more, 30% or more 50% or more. In certain embodiments, the fine dry particulate active is added directly to the delivery vehicle (i.e., the fine dry particulate active is not wetted prior to combining/mixing with the delivery vehicle). In other words, the fine dry particulate active and the delivery vehicle are combined to form the topical composition.

The delivery vehicle (i.e., topical delivery component) refers to that portion of the topical composition that is not the fine dry particulate active. Delivery vehicles of interest include, but are not limited to, compositions that are suitable for applications via one or more of oral, topical, implantation, ocular, aural, rectal, vaginal, etc., routes. In certain embodiments, the vehicle is formulated for application to a topical region or surface of a subject, such as a keratinized skin surface. The subject compositions may be formulated as stable solutions or suspensions of the components, e.g., in an aqueous solvent. Where desired, the components may be combined with one or more carrier materials to form a solution, suspension, gel, lotion, cream, ointment, aerosol spray, roll-on, foam products, mousses, powders, sticks, or the like, as desired. Of interest in certain embodiments are aqueous delivery vehicles, i.e. aqueous vehicles that include a certain amount of water. Examples of aqueous vehicles include hydrogel vehicles, sprays, serums, etc.

The topical composition may also contain other physiologically acceptable excipients or other minor additives, particularly associated with organoleptic properties, such as fragrances, dyes, buffers, cooling agents (e.g. menthol), coating materials or the like. The excipients and minor additives will be present in conventional amounts, e.g., ranging from about 0.001% to 5%, such as 0.001-2%, by weight, and in some instances not exceeding a total of 10% by weight.

Lotions (as well as other topical formulations) of interest may include one or more of the following components: Water, Viscosity modifiers, Humectants, Vegetable oils and hydrogenated vegetable oils, Emollients, Conditioning Agents, Emulsifiers, Glyceryl Esters of Fatty Acids, Silicone, C1-C30 monoesters and polyesters of sugar, Conditioning Agents, Preservatives, etc. Depending on the topical formulation, additional components of interest include: Abrasives, Absorbents, Antimicrobial and antifungal agents, Astringents, Anti-Acne agents, Anti-wrinkle agents, Anti-oxidants, Antimicrobials, Binders, Biological actives, Buffering actives, Bulking actives, Chelating agents, Chemical additives, External analgesics, Film former agents, Opacifying agents, pH adjusters, Reducing agents, Colorants, Fragrances, Cosmetic Soothing Agents, Tanning actives & accelerators, Skin lightening/whitening agents, Sunscreens, Surfactants, Skin Conditioning Agents, Vitamins, etc.

As indicated above, of interest in certain embodiments are semi-solid delivery compositions, such as gels, creams and ointments. Such compositions may be mixtures of (in addition to the active agent) water, water soluble polymers, preservatives, alcohols, polyvalent alcohols, emulsifying agents, wax, solvents, thickeners, plasticizers, pH regulators, water-retaining agents and the like. Furthermore, such compositions may also contain other physiologically acceptable excipients or other minor additives, such as fragrances, dyes, buffers, coating materials or the like.

Also of interest are solid formulations, such as topical patch formulations. Topical patch formulations may vary significantly. Topical patch formulations may include an active agent layer, a support and a release liner. The active agent layer may include physiologically acceptable excipients or other minor additives, such as fragrances, dyes, buffers, coating materials or the like. The support may be made of a flexible material which is capable of fitting in the movement of human body and includes, for example, plastic films, various non-woven fabrics, woven fabrics, spandex, and the like. Various inert coverings may be employed, which include the various materials which may find use in plasters, described below. Alternatively, non-woven or woven coverings may be employed, particularly elastomeric coverings, which allow for heat and vapor transport. These coverings allow for cooling of the pain site, which provides for greater comfort, while protecting the gel from mechanical removal. The release liner may be made of any convenient material, where representative release films include polyesters, such as PET or PP, and the like.

When present in the delivery vehicle, a high weight percentage of the active agent of the initial fine dry particulate composition may remain associated with the calcium particles. In some instances, the weight percentage that remains associated with the calcium particles (and therefore is not free in the delivery vehicle) is 40% or more, such as 50% or more, including 60% or more, e.g., 70% or more. Active agent that remains associated with the calcium particles may be carried along with the particles into the skin for delivery in the acidic environment of the skin.

### Utility

Topical formulations of the invention find use in methods of delivering active agents to a topical location of a subject, where the topical location may be a skin surface location or a mucosal location. In delivering active agents to a topical location of a subject, formulations of the invention may deliver the active agent at least into an epidermal location that is beneath the skin surface of a subject. As such, embodiments of the invention include methods of delivering active agent/calcium particle complexes into the stratum corneum of a subject, where the methods may result in delivery of the complexes into the deep stratum corneum and/or dermis of a subject. By “deep stratum corneum” is meant a region that is 1 or more cell layers below the skin surface, such as 2 or more, e.g., 5 or more cell layers below the skin surface, including 10 or more cell layers below the skin surface. In some instances, the active agent/calcium particle complexes are delivered to region of the stratum corneum that is 2 μm or more such as 5 μm or more and including 15 μm or more below the surface of the skin.

Upon reaching their target dermal location, the active agent/calcium particle complexes may begin to release their active agent “payload” and break down (e.g., via dissolution caused by pH gradient of the skin), as the uniform, rigid, spherical, nanoporous particles dissolve under acidic conditions, e.g., conditions of pH 5.5 or lower, such as 5 or lower, including 4.0 or lower, such as the physiological acidic conditions of the stratum corneum. The time required for dissolution of particles in the stratum corneum may vary, and in certain embodiments ranges from a few minutes up to several days, such as 1 minute to 24 hours, such as 10 minutes to 12 hours and including 30 minutes to 3 hours, over which time period active agent is released from the fine particulate dry active. The proportion of active agent that is released from the active agent/calcium particle complexes may vary, and in certain instances is 0.01% or more, such as 0.1% or more, including 1% or more, such as 10% or more, including 50% or more, 75% or more, including up to 100% (w/w).

Methods of the invention may therefore result in delivery of an active agent at least into the stratum corneum of a subject. Additional target locations of the body of interest include additional epidermal regions, such as but not limited to the stratum lucidum, stratum granulosum, stratum spinusom, stratum basale and dermis. In certain embodiments, the active agent is delivered to a region of the dermis. In certain embodiments, the active agent is delivered to a region below the dermis, e.g., into sub-cutaneous tissues.

In practicing methods of the invention, a topical formulation is applied to a topical region of a subject and maintained at the topical region for a period of time sufficient to result in the desired delivery of active agent to the subject, as described above. The topical region is, in certain embodiments, a keratinized skin region. The keratinized skin region, including hair follicles, sweat glands and sebaceous glands, may be present at a variety of locations, e.g., limbs, arms, legs; torso, e.g., chest, back, stomach; head, e.g., neck, face; etc. In certain embodiments, the region will be a head region, such as a facial region, e.g., forehead, occipital region, around the mouth, etc. The topical region to which the composition is applied may vary with respect to area, ranging in certain embodiments from 1 mm² to 20,000 cm² or more, such as from 1 to 50 cm², and including from 3 to 10 cm².

Following application, the topical formulation is maintained at the site of application for a period of time sufficient for a desired therapeutic outcome to occur, e.g., amelioration of a symptom(s) of interest, reducing dryness. The period of time may vary, and in certain embodiments ranges from instantaneously up to several days, such as 1 min to 24 hours or longer, such as from 30 min to 12 hours and including from 1 hour to 12 hours or longer.

In practicing the methods of the invention, a subject may be administered a single dose or two or more doses over a given period of time. For example, over a given treatment period of one month, 1 or more doses, such as 2 or more doses, 3 or more doses, 4 or more doses, 5 or more doses, etc., may be administered to the subject, where the doses may be administered weekly or daily or even multiple times per day, with a holiday period in between, e.g., where the holiday period may vary, e.g., 4 hours, 6 hours, 12 hours, 1 day, 3 days, 7 days, etc.

The subject methods and compositions may be used in a variety of different kinds of animals, where the animals are typically “mammals” or “mammalian,” where these terms are used broadly to describe organisms which are within the class mammalia, including the orders carnivore (e.g., dogs and cats), rodentia (e.g., mice, guinea pigs, and rats), lagomorpha (e.g., rabbits) and primates (e.g., humans, chimpanzees, and monkeys). In certain embodiments, the subjects or patients are humans.

The subject topical formulations find use in applications where it is desired to deliver an adenosine active agent to a subject. In certain embodiments, the subject topical formulations are employed in the treatment of a skin condition. By “treatment” is meant that at least an amelioration of the symptoms associated with the condition afflicting the subject is achieved, where amelioration is used in a broad sense to refer to at least a reduction in the magnitude of a parameter, e.g. symptom, associated with the condition being treated. As such, treatment also includes situations where the condition, or at least symptoms associated therewith, are completely inhibited, e.g. prevented from happening, or stopped, e.g. terminated, such that the subject no longer suffers from the condition, or at least the symptoms that characterize the condition. In certain embodiments a subject may be diagnosed for the presence of the disease condition, such that the topical formulations are provided to a subject known to be suffering from the disease condition.

Practice of methods of the invention can enhance result in the improvement in skin, when there is a noticeable decrease in noticeable decrease in the amount of wrinkling, roughness, dryness, laxity, sallowness, or pigmentary mottling of the treated skin. Methods of measuring improvements in skin condition are well known in the art (see, e.g., Olsen et al., J. Amer. Acad. Dermatol. 26:215-24, 1992), and can include subjective evaluations by the patient or a second party, e.g., a treating physician. Objective methods can include skin topography measurements, such as those described in Grove et al., J. Amer. Acad. Dermatol. 21:631-37 (1989). In skin topography measurements, silicone rubber replicas are made of a small area of skin, e.g., a 1 cm diameter circular area. The silicone rubber replicas capture fine lines and wrinkles on the skin. These specimens are then analyzed using computerized digital image processing to provide an objective measurement of the skin's topography. Skin topography measurements generated following digital-image processing can be measured using the values Rₐ and R_{z} as described in Olsen et al., J. Amer. Acad. Dermatol. 37:217-26, 1997, where Rₐ represents the area of deviation of skin surface features above and below an average central line, and R_{z} represents the difference between the maximum and minimum heights in five equal segments of the skin surface profile. A statistically significant decline (e.g., P<0.05) in Rₐ and R_{z} values in skin treated according to the presence invention compared to untreated skin indicates an improvement in skin, as is achieved by practicing the methods of the invention.

The following examples are offered by way of illustration and not by way of limitation.

### EXPERIMENTAL

### I. Preparation of Fine Dry Adenosine Particulate Composition

### A. Protocol 1

0.1 g of adenosine and 0.1 g of nanoporous calcium phosphate particles (Nanoporous calcium phosphate particles having an average diameter of 2 μm used in the following examples were obtained from Laboratory Skin Care® (San Carlos, Calif.)) were mixed with 5 ml of water. Lactic acid was used to neutralize the solution to 6.8±0.1. A Buchi R-215 Rotovapor was used to completely dry off the solvent to produce fine dry adenosine particulate composition.

### B. Protocol 2

0.1 g of adenosine was mixed with 5 ml of water and 5 ml of Ethanol. The mixture was heated to 60° C. until adenosine was dissolved. To the solution was added 0.1 g of nanoporous calcium phosphate particles (Nanoporous calcium phosphate particles having an average diameter of 2 μm used in the following examples were obtained from Laboratory Skin Care® (San Carlos, Calif.)) A Buchi R-215 Rotovapor was used to completely dry off the solvent to produce fine dry adenosine particulate composition.

### C. Protocol 3

0.107 g of adenosine and 0.893 g of nanoporous calcium phosphate particles (Nanoporous calcium phosphate particles having an average diameter of 2 μm used in the following examples were obtained from Laboratory Skin Care® (San Carlos, Calif.)) were mixed with 5.35 ml of water. Lactic acid was used to neutralize the solution to 6.8±0.1. A Buchi R-215 Rotovapor was used to completely dry off the solvent to produce fine dry adenosine particulate composition.

### II. Characterization

### A. Stability

### 1. Shelf-Life

Shelf life study of fine dry particulate adenosine composition was conducted. Samples were stored at room temperature, 40° C. and 50° C. After incubation, Adenosine from fine dry particulate adenosine compositions was extracted by HPLC water and analyzed by reverse-phase HPLC for adenosine. The quantification of adenosine was achieved by external standard calibration. Adenosine (spectrum chemical) alone was placed at the same incubation condition as control.

Particulate adenosine was stable at room temperature, and at 40° C. for three months, and at 50° C. for one month. No change in pH, color, appearance, was observed.

### 2. Formulation Stability

Fine dry particulate adenosine composition (1% adenosine) was added to a base cream (See Table 1, below) and mixed until uniform. A sample of the adenosine cream was stored at room temperature, 40° C., and 50° C. Adenosine from the incubated adenosine cream was extracted by HPLC water, and analyzed by reverse-phase HPLC for adenosine. The quantification of adenosine was achieved by external standard calibration. Adenosine (spectrum chemical) alone was place at the same base cream incubation condition as control.

Fine dry particulate adenosine composition in base cream was stable at room temperature, at 40° C. for three months, and at 50° C. for one month. No change in pH, color, appearance, or viscosity was observed.

TABLE 1
Fine Dry particulate Adenosine composition in base cream

| Raw Material | % w/w |
|---|---|
| D.I. Water | 83.687% |
| Xanthan Gum | 0.300% |
| Glycerin | 1.000% |
| Caprylic/Capric Triglyceride | 3.000% |
| Stearyl Alcohol | 2.000% |
| Cetyl Alcohol | 2.000% |
| Lipomulse 165 (INCI: Glyceryl Stearate, PEG-100 Stearate) | 1.000% |
| Cyclopentasiloxane | 3.000% |
| Euxyl PE 9010 (INCI: Phenoxyethanol, Ethylhexylglycerin) | 1.000% |
| Sepiplus 400 (iNCI: Polyacrylate-13, Polyisobutene, | 1.000% |
| Polysorbate 20) | |
| Triethanolamine | 0.013% |
| Fine Dry particulate Adenosine composition (INCI: | 2.000% |
| Adenosine, Hydroxyapatite) | |
| Total | 100.000% |

### B. Dermal Delivery

### 1. Procedure:

Franz cell 24-hr flow through as described in J Pharm Sci. 1992 December; 81(12):1153-6. In vitro release of nitroglycerin from topical products by use of artificial membranes. Wu S T, Shiu G K, Simmons J E, Bronaugh R L, Skelly J P. Tissue: Human abdominoplasty skin

Vehicle: Cream (see Table 1, above).

Sampling: Receptor phase

Analysis: HPLC

The separation was achieved using a Phenomenex Gemini™ C18 column (4.5 mm id×50 mm, 3 μm) and a mobile phase of gradient run of methanol and water from (0% methanol to 80% methanol in 5 min) and a flow rate of 1 ml/min. The eluent was monitored for adenosine at 260 nm. The quantification of adenosine was achieved by external standard calibration.

Control: adenosine alone (spectrum chemical) in the same base cream.

### 2. Results

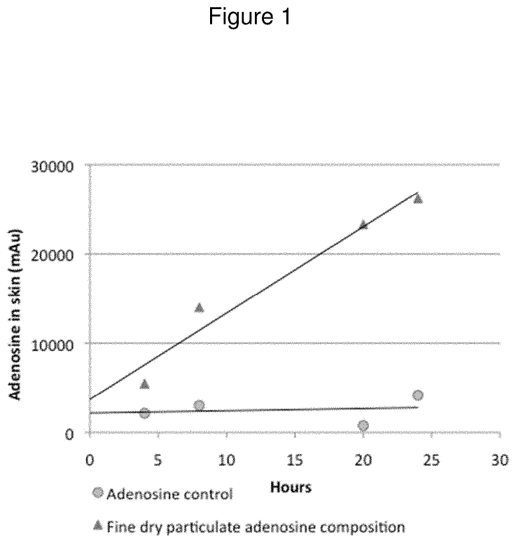

Adenosine skin penetration with fine dry particulate adenosine composition was observed to be 6 fold greater than that of control. See FIG. 1.

Various aspects of the invention are further described in the following clauses:
Clause 1. A method comprising:
   (a) combining:
      (i) nanoporous calcium particles comprising a porous structure that defines an internal space; and
      (ii) an adenosine active agent;
      in the presence of an aqueous solvent to produce a mixture; and
   (b) removing the aqueous solvent from the mixture to produce a fine dry particulate adenosine composition.
Clause 2. The method according to Clause 1, wherein the nanoporous calcium particles are nanoporous calcium phosphate particles.
Clause 3. The method according to Clause 2, wherein the nanoporous calcium phosphate particles are uniform, rigid and spherical.
Clause 4. The method according to Clause 3, wherein the uniform, rigid, spherical, nanoporous calcium phosphate particles are ceramic.
Clause 5. The method according to Clause 4, wherein the uniform, rigid, spherical, nanoporous calcium phosphate particles are dimensioned to penetrate human skin.
Clause 6. The method according to Clause 5, wherein the uniform, rigid, spherical, nanoporous calcium phosphate particles have a diameter ranging from 1 to 10 μm.
Clause 7. The method according to Clause 6, wherein the uniform, rigid, spherical, nanoporous calcium phosphate particles have a diameter of 2 μm or less.
Clause 8. The method according to Clause 1, wherein the uniform, rigid, spherical, nanoporous calcium phosphate particles comprise pores ranging in size from 2 nm to 100 nm.
Clause 9. The method according to Clause 1, wherein the adenosine active agent is selected from the group consisting of adenosine monophosphate, adenosine diphosphate and adenosine triphosphate and combinations thereof.
Clause 10. The method according to Clause 1, wherein the particles and adenosine active agent are combined under negative pressure.
Clause 11. The method according to Clause 1, wherein the solvent is removed from the mixture under negative pressure.
Clause 12. The method according to Clause 1, wherein the method further comprises stabilizing the fine dry particulate active.
Clause 13. The method according to Clause 12, wherein the stabilizing comprises associating the fine dry particulate active with a coating material.
Clause 14. The method according to Clause 13, wherein the coating material comprises a wax.
Clause 15. A fine dry particulate adenosine composition that includes an adenosine active agent(s) present inside of the pores of nanoporous calcium particles and/or on the surface of the particles and/or loosely associated with the particles.
Clause 16. The fine dry particulate adenosine composition according to Clause 15, wherein the weight percentage of adenosine active agent in the composition ranges from 0.001 up to 100.
Clause 17. The fine dry particulate adenosine composition according to Clause 15, wherein the nanoporous calcium particles are nanoporous calcium phosphate particles.
Clause 18. The fine dry particular adenosine composition according to Clause 17, wherein the nanoporous calcium phosphate particles are uniform, rigid, spherical, nanoporous calcium phosphate particles are ceramic.
Clause 19. The fine dry particulate adenosine composition according to Clause 18, wherein the uniform, rigid, spherical, nanoporous calcium phosphate particles are ceramic.
Clause 20. The fine dry particulate adenosine composition according to Clause 19, wherein the uniform, rigid, spherical, nanoporous calcium phosphate particles are dimensioned to penetrate human skin.
Clause 21. The fine dry particulate adenosine composition according to Clause 20, wherein the uniform, rigid, spherical, nanoporous calcium phosphate particles have a diameter ranging from 1 to 10 μm.
Clause 22. The fine dry particulate adenosine composition according to Clause 21, wherein the uniform, rigid, spherical, nanoporous calcium phosphate particles have a diameter of 2 μm or less.
Clause 23. The fine dry particulate adenosine composition according to Clause 17, wherein the nanoporous calcium phosphate particles comprise pores ranging in size from 2 nm to 100 nm.
Clause 24. The fine dry particulate adenosine composition according to Clause 15, wherein the adenosine active agent is selected from the group consisting of adenosine monophosphate, adenosine diphosphate and adenosine triphosphate and combinations thereof.
Clause 25. The fine dry particulate adenosine composition according to Clause 15, wherein composition comprises a coating material.
Clause 26. The fine dry particulate adenosine composition according to Clause 25, wherein the coating material comprises a wax or butter.
Clause 27. A topical formulation comprising:
   a fine dry particulate adenosine composition according to any of Clauses 15 to 26; and
   a topical delivery vehicle.
Clause 28. The topical composition according to Clause 27, wherein the topical delivery vehicle is an aqueous topical delivery vehicle.
Clause 29. A method of delivering an adenosine active agent to a subject, the method comprising:
   applying a topical formulation comprising:
      (a) a fine dry particulate adenosine composition according to any of Clauses 15 to 26; and
      (b) a topical delivery vehicle;
   to a topical region of the subject to deliver the adenosine active agent to the subject.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A fine dry particulate adenosine composition that includes an adenosine active agent(s) present inside of the pores of nanoporous calcium particles and/or on the surface of the particles and/or loosely associated with the particles.

2. The fine dry particulate adenosine composition according to claim 1, wherein the weight percentage of adenosine active agent in the composition ranges from 0.001 up to 100.

3. The fine dry particulate adenosine composition according to claim 1, wherein the nanoporous calcium particles are nanoporous calcium phosphate particles.

4. The fine dry particular adenosine composition according to claim 3, wherein the nanoporous calcium phosphate particles are uniform, rigid, spherical, nanoporous calcium phosphate particles.

5. The fine dry particulate adenosine composition according to claim 4, wherein the uniform, rigid, spherical, nanoporous calcium phosphate particles are ceramic.

6. The fine dry particulate adenosine composition according to claim 4, wherein the uniform, rigid, spherical, nanoporous calcium phosphate particles have a diameter ranging from 1 to 10 μm.

7. The fine dry particulate adenosine composition according to claim 6, wherein the uniform, rigid, spherical, nanoporous calcium phosphate particles have a diameter of 2 μm or less.

8. The fine dry particulate adenosine composition according to claim 1, wherein the nanoporous calcium particles comprise pores ranging in size from 2 nm to 100 nm.

9. The fine dry particulate adenosine composition according to claim 1, wherein the adenosine active agent is selected from the group consisting of adenosine monophosphate, adenosine diphosphate and adenosine triphosphate and combinations thereof.

10. A topical formulation comprising:
a fine dry particulate adenosine composition according to claim 1; and
a topical delivery vehicle.

11. The topical composition according to claim 10, wherein the topical delivery vehicle is an aqueous topical delivery vehicle.

12. A method of delivering an adenosine active agent to a subject, the method comprising:
applying a topical formulation comprising:
(a) a fine dry particulate adenosine composition according to claim 1; and
(b) a topical delivery vehicle;
to a topical region of the subject to deliver the adenosine active agent to the subject.

13. A method comprising:
(a) combining:
(i) nanoporous calcium particles comprising a porous structure that defines an internal space; and
(ii) an adenosine active agent;
in the presence of an aqueous solvent to produce a mixture; and
(b) removing the aqueous solvent from the mixture to produce a fine dry particulate adenosine composition.

14. The method according to claim 13, wherein the method is a method of making a fine dry particulate adenosine composition that includes an adenosine active agent(s) present inside of the pores of nanoporous calcium particles and/or on the surface of the particles and/or loosely associated with the particles.

15. The method according to claim 14, wherein the particles and adenosine active agent are combined under negative pressure.
